(19) United States
(12) Patent Application Publication
(10) Pub. No.: US 2010/0080541 A1
(43) Pub. Date: Apr. 1, 2010

(54) Process of creating and applying live tree fragrance and aroma for artificial christmas trees

(76) Inventors: Gene Arthur De La Zerda, Eagle Point, OR (US); Garry M. Burgess, Beaverton, OR (US)

Correspondence Address: Gene A. De La Zerda, 195 Leafwood Drive, Eagle Point, OR, 97524, US

(21) Appl. No.: 12/378,339

(22) Filed: Feb. 13, 2009

Related U.S. Application Data
(60) Provisional application No. 61/100,605, filed on Sep. 26, 2008.

(51) Int. Cl. A61L 9/03

(52) U.S. Cl. 392/395

(57) ABSTRACT

A process of creating and applying live tree fragrance and aroma for artificial Christmas trees. This process utilizes an all natural organic fluid extracted from the conifer tree family. The conifer needle oil is heated to its vaporization point within the vapor dispenser to produce its true fragrance. Then it is gently fanned to disperse the fragrance and aroma around an artificial Christmas tree.

### CROSS-REFERENCE TO RELATED APPLICATIONS

This application claims the benefits of provisional patents Ser. No. 61/078,210 Filed Jul. 3, 2008 and Ser. No. 61/100,605 filed Sep. 26, 2008 by the present inventors.

### FEDERALLY SPONSORED RESEARCH

Not Applicable

### SEQUENCE LISTING OR PROGRAM

Not Applicable

### BACKGROUND

1. Field

This application relates to a live tree fragrance and aroma for artificial Christmas trees.

2. Prior Art

Since the inception of artificial Christmas trees over 75 years ago, there has not been a safe non-toxic, live tree fragrance or aroma available to consumers. Typically, pine oil or pine fragrance have been the only products available to give this scent and aroma. Both these products are toxic and highly flammable.

Artificial Christmas trees are now more realistic and beautiful than ever, but the lack of fragrance and aroma is still the major drawback in their use. This process solves the problem of artificial Christmas trees not having a live tree fragrance and aroma in a safe and non-flammable way.

Stanley U.S. patent 2004/0197221 A1 Oct. 7, 2004 shows a hollow artificial tree and hollow branches in which fragrance is pumped from a reservoir through the tree itself. This type of system is wasteful.

Hunter U.S. Pat. No. 6,371,453 Apr. 16, 2002 shows a steam diffusor to produce a fragrance. This steam vaporizing system uses dry plant material to extract essential oils and fragrance. Many of the essential oils contain volatile and hazardous substances.

Spector U.S. Pat. No. 5,569,511 Oct. 29, 1996 has decorated balls and ornaments to hang on artificial Christmas trees to exude a fragrance based on pine needles. However, these products have primarily been alcohol or solvent based liquids that also produce toxic and flammable fumes.

Davis U.S. Pat. No. 5,455,750 Oct. 3, 1995 shows an artificial Christmas tree which incorporates a scent production element. This system, again, incorporates a hollow trunk and tree system for pressurized production of aroma and scent.

Unlike these expensive and complicated systems, our vapor dispenser is small and inexpensive. Its design enables the dispenser to be installed on or near any artificial Christmas tree.

### DRAWINGS—FIG. 1—FLOWCHART

The flowchart explains the processes involved in creating and applying a live tree fragrance to artificial Christmas trees.

In the flowchart, closely related procedures have the same number but different alphabetic suffixes.

FIG. 1A shows conifer needle oil derived from any of the approximately 630 species of the conifer tree family. Conifers include cedars, douglas fir, cypresses, firs, junipers, kauris, larches, pines, redwoods, spruces, and yews.

FIG. 1B shows needle oil used in its pure form, or blended with other species within the conifer family. For example: Canadian fir (Abies balsamea) needle oil produces a strong Douglas fir tree scent. Blending Canadian fir (Abies balsamea) needle oil and Eastern Spruce (Picea mariana) needle oil will produce a spruce tree scent. Blending Canadian fir (Abies balsamea) needle oil and Sylvestris needle oil will produce a scotch pine aroma.

FIG. 1C shows blended conifer needle oil or a synthetically engineered compound used to produce a tree like fragrance or aroma.

FIG. 1D shows the vaporization process achieved by a flame proof heating resistor to enhance the fluid to its full fragrance potential.

FIG. 1E shows vaporized fluid dispersed into air utilizing a variable speed fan within vapor dispenser.

FIG. 1F shows vapor dispenser mounted on artificial Christmas tree or plugged into a nearby 110 volt wall socket.

DRAWINGS—FIG. 2

FIG. 2—Vapor Dispenser

FIG. 2a shows dimensional front view of vapor dispenser and fluid reservoir.

FIG. 2b shows side view cutaway of vapor dispenser's internal parts.

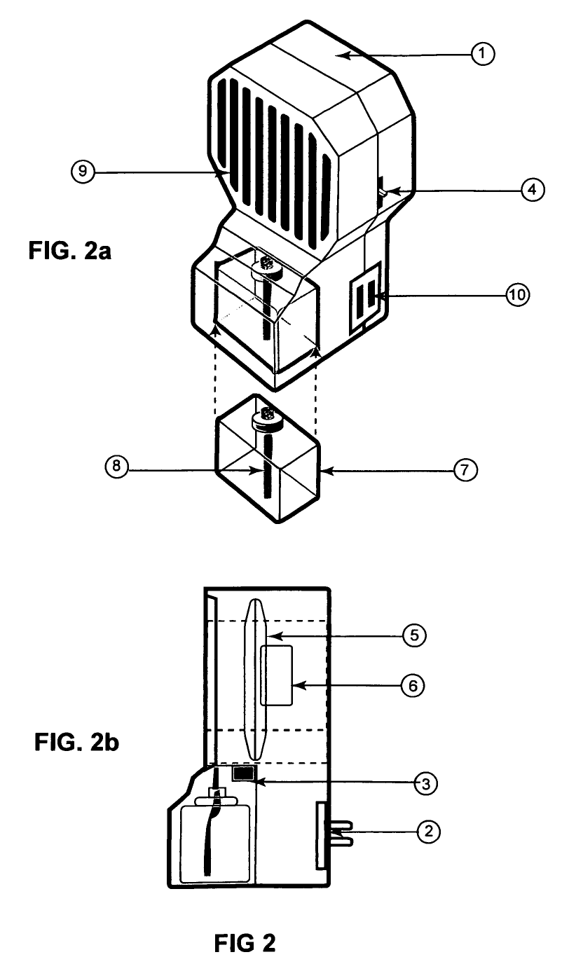

### REFERENCE NUMERALS FOR FIG. 2a AND 2b

1 Plastic Housing

2 Electrical Blades for 110 volt operation

3 Heating Element

4 Variable Speed Switch

5 Fan

6 Variable Speed Fan Motor

7 Fluid Reservoir

8 Fiberous Wick

9 Louvers

10 Electrical Outlet

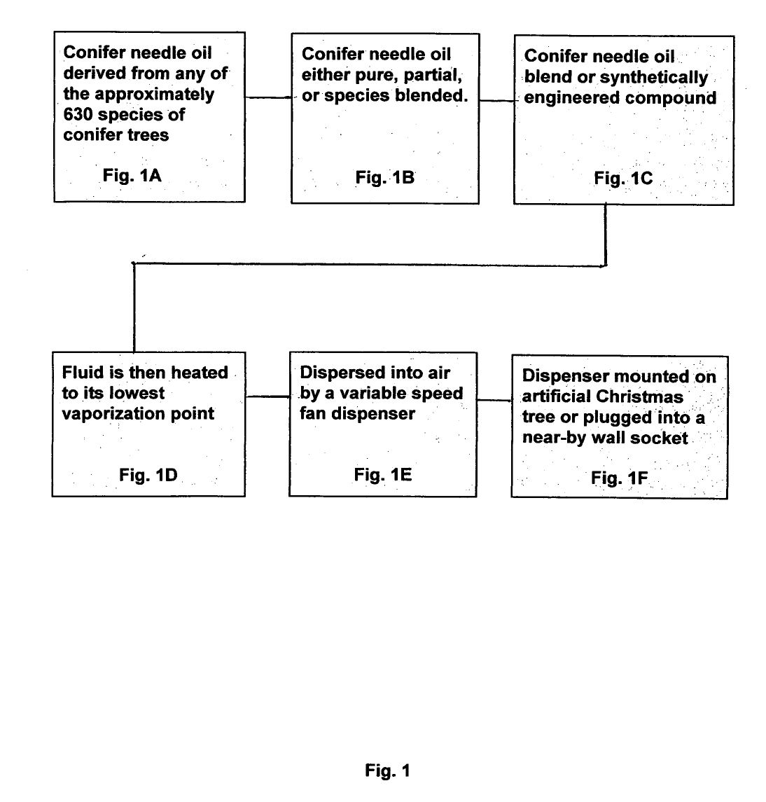

### Embodiment—FIG. 1

The flowchart explains the processes involved in creating and applying a live tree fragrance and aroma to any artificial Christmas tree. Conifer needle oil is derived from needles only of the conifer tree family. These include approximately 630 species within the plant kingdom. Conifer needle oil may be used pure or blended with other species within the conifer family.

Conifer needle oil may also be blended with resinoids, or synthetic compounds to prolong or produce similar aromatic effects.

Conifer needle oil must be heated to a point of vaporization to enhance the fragrance from its static state to its full aromatic potential. Once vaporized, it is gently fanned to disperse the fragrance into the air. The vapor dispenser would either be mounted on the artificial Christmas tree and 110 volt power applied to it, or plugged into a wall socket near by.

### Embodiment—FIG. 2

FIG. 2 shows the conifer needle oil dispenser with main elements. The housing I is constructed of PP plastic which is flame-proof. The heating element 3 is an electronic flame-proof resistor with temperature raising capabilities of vaporizing the needle oil. The switch 4 controls the fan 5. The fan 5 and Fan Motor 6 consists of variable speeds. The Fluid Reservoir 7 holds the conifer needle oil and the wick 8 to bring fluid up to the heating element 3 for dispersal through the Louvers 9. The vapor dispenser operates on 110 voltage through the electrical blades 2. Electrical outlet 10 may be used as an alternative electrical outlet.

### Operation

Conifer needle oil is a plant extract derived from needles only of any of the approximately 630 species of conifer trees. This fluid in its static state will not produce the aromatic fragrance of their parent species. This fluid, whether pure, partial, or species blended, must be heated to its lowest vaporization point to enhance its full aroma and potential. The fan mounted above the heating element gently disperses the vaporized fluid near the artificial Christmas tree. The variable speed fan is slowed for a small room and increased for rooms of larger size.

### Advantages

(a) Using conifer needle oil in this manner creates a non-toxic, non-flammable tree like fragrance and aroma.

(b) Conifer needle oil when heated to its lowest vaporization point is invigorating and emotionally uplifting.

(c) This process creates an inexpensive and safe tree-like fragrance and aroma that could either be used during the holidays or year-round.

### Alternate Embodiments

With current technology, this product can be manufactured in at least two separate forms: environmentally safe utilizing this all-natural organic fluid; or synthetically for cost reduction and fluid longevity. However, most synthetic fragrances are currently based on petroleum, alcohol, or shale oil. These synthetics are toxic and flammable.

### Conclusion and Scope

The method and usage of conifer needle oils to produce a live tree fragrance and aroma for artificial Christmas trees should not be limited to just the holiday season. This non-toxic, non-flammable fluid is invigorating and uplifting and may be used in homes year-round. With approximately 630 conifer species, the combinations of conifer needle oil blends are nearly endless making this a new field of completely organic fragrances.

We claim:

1. A method for creating and applying live-tree fragrances and aromas for artificial Christmas trees comprising:
a. utilizing needle oil from any of the approximately 630 species of conifer trees to create said live-tree fragrances and aromas.
b. vaporizing and gently fanning conifer needle oil to provide said live tree fragrances and aromas.
c. using said conifer needle oil or a synthetic fragrance to mimic or match said conifer needle oil either pure, partial, or species blended.
d. using said conifer needle oil to produce non-toxic, non-flammable fragrances and aromas.
e. dispersing said fragrances and aromas by vapor dispenser on or near artificial Christmas trees.

2. A vapor dispenser comprising:
a. a plastic housing.
b. a heating element.
c. a variable speed fan.
d. a variable speed control switch.
e. a plastic fluid reservoir.
f. a wick of fiberous material.
g. electrical blades for 110 volt operation
h. electrical outlet
i. louvers for air passage
